(19) United States
(12) Patent Application Publication
(10) Pub. No.: US 2026/0007306 A1
(43) Pub. Date: Jan. 8, 2026

(54) MEDICAL DEVICES AND RELATED SYSTEMS AND METHODS

(71) Applicants: Boston Scientific Scimed, Inc., Maple Grove, MN (US); Boston Scientific Medical Device Limited, Galway (IE)

(72) Inventors: Benjamin CLEVELAND, Bellingham, MA (US); John SUBASIC, Stow, MA (US); Nitesh Ghananil BAVISKAR, Kalyan West, Maharashtra (IN); Snehal PATIL, Pune, Maharashtra (IN); Shrikant Vasant RAUT, Pune, Maharashtra (IN); Katie KNOWLES, Providence, RI (US)

(73) Assignees: Boston Scientific Scimed, Inc., Maple Grove, MN (US); Boston Scientific Medical Device Limited, Galway (IE)

(21) Appl. No.: 19/259,610

(22) Filed: Jul. 3, 2025

Related U.S. Application Data
(60) Provisional application No. 63/668,417, filed on Jul. 8, 2024.

(51) Int. Cl. A61B 1/005

(52) U.S. Cl. CPC A61B 1/0057

(57) ABSTRACT

Medical devices and related methods are described, including a medical device that includes a shaft including a distal articulating portion; and a handle coupled to the shaft. The handle may include a first actuator moveably coupled to a handle body of the handle, and the first actuator may be configured to initiate bending of the distal articulating portion in a first direction when the first actuator is translated along a first axis transverse from a central longitudinal axis of the medical device.

### CROSS-REFERENCE TO RELATED APPLICATION(S)

This application claims the benefit of priority to U.S. Provisional Application No. 63/668,417, filed on Jul. 8, 2024, which is incorporated by reference herein in its entirety.

### TECHNICAL FIELD

Various aspects of this disclosure relate generally to medical devices and methods of use thereof. In particular, medical devices and methods useful for procedures involving devices insertable into a patient.

### BACKGROUND

During medical procedures, such as endoscopy and/or ureteroscopy procedures, an operator may position a medical device at a target treatment site in a body lumen of a patient, and may articulate a shaft of the medical device by actuating one or more actuators of a handle of the medical device. For users with smaller hands, some actuators may be difficult to fully actuate to achieve full articulation of the shaft, and their smaller hands may prevent these users from effective operation of the medical device. For example, rotatable knob actuators may be more difficult for users with smaller hands to rotate through the full range of positions of the knob, and thus may reduce or prevent effective operation of the medical device. Additionally, some actuators require a user to position their hand in an uncomfortable position relative to a handle, and may increase user fatigue during operation of the medical device due to the position, size, and orientation of the actuator. Devices, systems, and methods of this disclosure may address some or all of these difficulties, and may address other problems in the art.

### SUMMARY

This disclosure relates to, among other things, medical devices and systems including one or more actuators, and related methods of use. Aspects of this disclosure may decrease the time to complete a procedure, such as removal of a kidney stone, from a patient's body. Further, for example, aspects of this disclosure may reduce the level of skill of a practitioner sufficient to operate a medical device, and/or may prevent damage to one or more devices during a procedure. Aspects of this disclosure may facilitate preventing user fatigue during a medical operation. Although primarily discussed in the context of kidney stone removal procedures, any of the systems, devices, and features thereof discussed herein may be used or adapted for use in other parts of the body to remove other materials or obstructions, or be used in other ways. The medical devices and systems disclosed herein in various examples may include one or more of the features or components described in connection with any of the other examples.

In some aspects, a medical device comprises a shaft including a distal articulating portion; and a handle coupled to the shaft. The handle comprises a first actuator moveably coupled to a handle body of the handle, and the first actuator is configured to initiate bending of the distal articulating portion in a first direction when the first actuator is translated along a first axis transverse from a central longitudinal axis of the medical device.

In other aspects, the medical device may include one or more of the following features. The handle may further comprise a second actuator moveably coupled to the handle body, and the second actuator may be configured to initiate bending of the distal articulating portion in a second direction when the second actuator is translated along a second axis transverse from a central longitudinal axis of the medical device, wherein the second direction is different from the first direction. The first actuator and the second actuator may be positioned at a proximal portion of the handle. The handle may further comprise an actuation member pivotably coupled to a body of the handle, and the first actuator abuts and/or is pivotably coupled to the actuation member. The actuation member may be cylindrical. A first articulation wire may be coupled to the actuation member, and the first articulation wire may extend longitudinally to a first portion of the distal articulating portion. A second articulation wire may be coupled to the actuation member, and the second articulation wire extends longitudinally to a second portion of the distal articulating portion. The handle may further comprise a third actuator positioned proximal of the first actuator and the second actuator.

In other aspects, the medical device may include one or more of the following features. The first actuator may include a first portion and an expanded distal portion, and the first portion is positioned within the handle body and at least a portion of the expanded distal portion is external of the handle body. The first portion (i) may be pivotably coupled to an actuation member or (ii) may include a rolling wheel which is configured to roll along a surface of the actuation member. Translation of the first actuator along the first axis may be configured to rotate the actuation member, and the first portion may be a cylindrical portion. The first actuator may be received within a first recessed portion of a body of the handle, and the second actuator may be received within a second recessed portion of the body of the handle. The handle may further comprise an actuation member coupled to a body of the handle at a first pivot point; and the first actuator may abut and/or be pivotably coupled to the actuation member within the handle body at a second pivot point, and the second actuator may abut and/or be pivotably coupled to the actuation member at a third pivot point on an opposite side of the first pivot point from the second pivot point. The second pivot point and the third pivot point may be longitudinally aligned with a longitudinal axis of the medical device when the medical device is in a neutral position. The first actuator and the second actuator may each be biased towards a neutral position.

In other aspects, a medical device may comprise a shaft including a distal articulating portion; and a first actuator. The first actuator is configured to initiate bending of the distal articulating portion in a first direction when the first actuator is translated along a first axis transverse from a central longitudinal axis of the medical device.

In other aspects, the medical device may include one or more of the following features. An actuation member may be coupled to and/or abut the first actuator, and the actuation member may be cylindrical and coupled to an articulation wire; wherein the articulation wire extends longitudinally through the shaft to the distal articulating portion. A second actuator may be coupled to and/or abutting the actuation member, and the second actuator may be configured to initiate bending of the distal articulating portion in a second direction when the second actuator is translated along a second axis transverse from a central longitudinal axis of the medical device, wherein the second direction is different from the first direction. The articulation wire may be a first articulation wire, and the medical device may further comprise a second articulation wire coupled to the articulation member and extending longitudinally through the shaft to the distal articulating portion.

In other aspects, a medical device comprises a shaft including a distal articulating portion; an articulation wire extending longitudinally through the shaft and coupled to a portion of the distal articulating portion; and a handle coupled to the shaft. The handle comprises a handle body, a first actuator, wherein the first actuator is configured to initiate bending of the distal articulating portion in a first direction when the first actuator is translated along a first axis, and an articulation member pivotably coupled to the first actuator and pivotably coupled to the handle body, wherein actuation of the first actuator is configured to rotate the articulation member and move the articulation wire.

### BRIEF DESCRIPTION OF FIGURES

The accompanying drawings, which are incorporated in and constitute a part of this application, illustrate exemplary aspects of this disclosure and together with the description, serve to explain the principles of the present disclosure.

FIG. 1 illustrates an exemplary medical device, in accordance with some aspects of this disclosure.

FIGS. 2A and 2B illustrate views of different portions of the medical device of FIG. 1, in accordance with some aspects of this disclosure.

FIGS. 3A and 3B illustrate side views of a portion of the medical device of FIG. 1 in different states of operation, in accordance with some aspects of this disclosure.

FIGS. 4A, 4B, and 4C side views of a hand in various states of operation of the medical device of FIG. 1, in accordance with some aspects of this disclosure.

### DETAILED DESCRIPTION

Reference will now be made in detail to aspects of this disclosure, examples of which are illustrated in the accompanying drawings. Wherever possible, the same or similar reference numbers will be used through the drawings to refer to the same or like parts.

It may be understood that both the foregoing general description and the following detailed description are exemplary and explanatory only and are not restrictive of the invention, as claimed. As used herein, the terms “comprises,” “comprising,” “includes,” “including,” or any other variation thereof, are intended to cover a non-exclusive inclusion, such that a process, method, article, device, or apparatus that comprises a list of elements does not include only those elements, but may include other elements not expressly listed or inherent to such process, method, article, device, or apparatus. The term “exemplary” is used in the sense of “example,” rather than “ideal.” Relative terms such as, for example, “about,” “substantially,” “approximately,” etc., are used to indicate a possible variation of +10% in a stated numeric value or range. The term “distal” refers to a portion farthest away from a user when introducing a device into a patient. By contrast, the term “proximal” refers to a portion closest to the user when placing the device into the patient. Proximal and distal directions are labeled with arrows marked “P” and “D”, respectively, throughout various figures. The term “transverse” is intended to mean extending or lying across; in a crosswise direction; and is not intended to mean extending across at a right or 90-degree angle.

Aspects of various devices are now described. Some aspects are described with reference to noninvasive procedures, such as urology procedures, wherein a sheath is advanced to a treatment site, and a capture device is extended from the sheath to engage an object at the treatment site. In urology procedures, for example, the sheath may be inserted into the urethra, moved through the bladder and ureter, and advanced into a calyx of a kidney; and the capture device may be extended distally from the sheath and/or a working channel of a ureteroscope or other medical device to engage one or more stones and/or stone fragments located in the calyx.

References to a particular type of medical procedure, such as a urology procedure; capture device, such as a basket or grasper; organ, such as a kidney; and/or object, such as a stone or stone fragment, are provided for convenience and not intended to limit this disclosure. Accordingly, the concepts described herein may be utilized for any analogous medical device or system.

The medical devices, systems, and methods discussed herein may facilitate visualization of a target site while breaking apart material (e.g. stones, etc.) during a procedure, may reduce the risk of injury to a patient's tissue during a procedure, may reduce the cost of a procedure, may reduce a user's fatigue during operation of a device, may facilitate effective operation of a device, may minimize the need for multiple devices during a procedure, and/or may increase accuracy of material removal, among other benefits.

Although ureteroscopes are referenced herein for illustration purposes, it will be appreciated that the disclosure encompasses any suitable medical device configured to allow an operator to access and view internal body anatomy of a subject (e.g., patient) and/or to deliver medical instruments, such as, for example, biopsy forceps, graspers, baskets, snares, probes, scissors, retrieval devices, lasers, and other tools, into the subject's body. The medical devices herein may be inserted into a variety of body lumens and/or cavities, such as, for example, the urinary tract or gastrointestinal tract. It will be appreciated that, unless otherwise specified, bronchoscopes, duodenoscopes, endoscopes, gastroscopes, endoscopic ultrasonography (“EUS”) scopes, colonoscopes, ureteroscopes, bronchoscopes, laparoscopes, cystoscopes, aspiration scopes, sheaths, catheters, or any other suitable delivery device or medical device may be used in connection with the features described herein.

According to some aspects of this disclosure, the medical device may include a handle coupled to a shaft; the shaft may extend to a distal articulating portion. The handle may include one or more actuators to actuate movement of the distal articulating portion. The distal articulating portion may include one or more cameras, one or more illuminators (e.g. LED lights, etc.), one or more elevator assemblies, and one or more working channels may extend through the shaft to distal articulating portion.

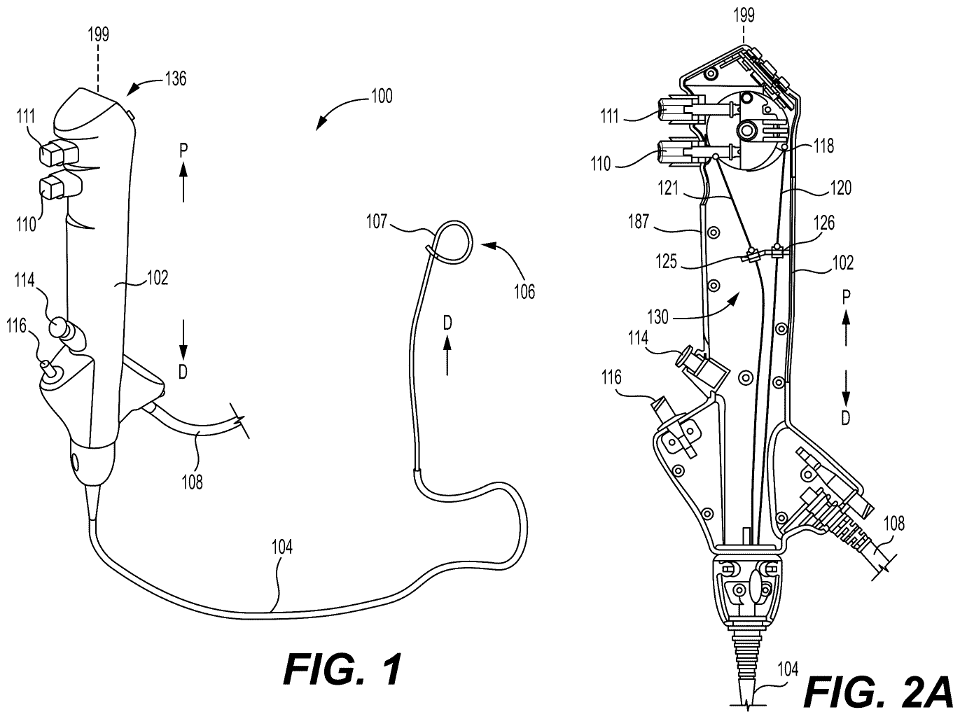

FIG. 1 illustrates an exemplary medical device 100 including a handle 102 and a shaft 104 extending distally from handle 102. Shaft 104 may include a distal articulating portion 106 including an articulation section 107 of the shaft 104. Handle 102 may include a handle body 187, one or more actuators 110, 111, 136, one or more fluid ports 114, and one or more working channel ports 116. An umbilicus 108 may be connected to handle 102, and umbilicus may electronically connect components of medical device 100 to a control unit or other electronic device. In some examples, umbilicus 108 may also supply fluid to one or more fluid channels of medical device 100 and/or suction (aspiration) to one or more channels of medical device 100.

Distal articulating portion 106 may include one or more cameras and one or more illuminators. One or more actuators 136 of handle 102 may control the one or more cameras and one or more illuminators of distal articulating portion 106. Although not clearly shown in FIG. 1, one or more actuators 136 may be push-button actuators, knobs, or any other type of actuator, such as an electronic screen touch interface or other actuator. Actuators 110, 111 may be push-button actuators and may be configured to control articulation (e.g. bending) of distal articulating portion 106 of shaft 104.

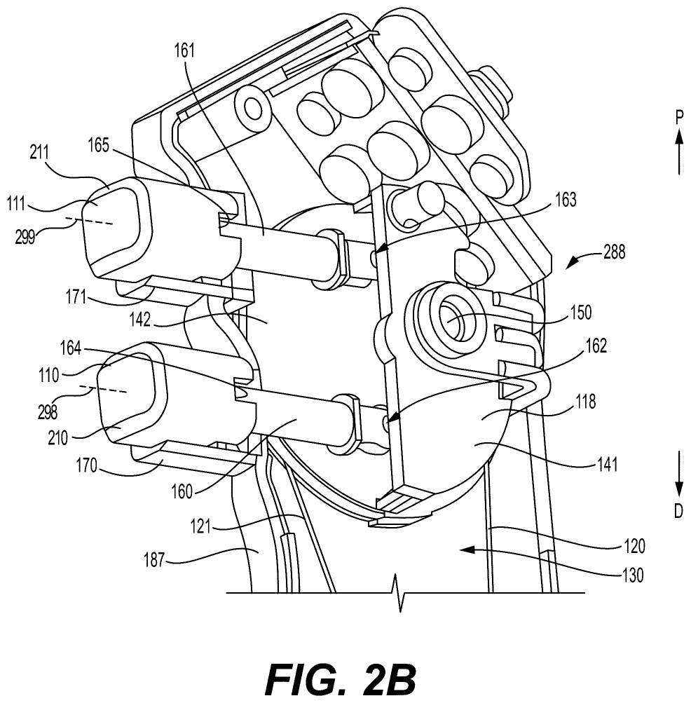

FIG. 2A illustrates a side view of handle 102 with a portion of handle 102 removed to expose interior components of handle 102. Actuators 110, 111 may control the movement of articulation wires 120, 121; and each articulation wire 120, 121 may extend through handle 102 and through shaft 104 to distal articulating portion 106 of shaft 104. An interior portion 130 of handle 102 may include articulation wire guides 125, 126, which may be ring-shaped apertures extending outward from an interior wall of the interior portion 130, and each wire guide 125, 126 may be configured to receive an articulation wire 120, 121 to guide the articulation wire 120, 121 through a portion of handle 102. Each of articulation wires 120, 121 may be coupled to an actuator member 118. Actuator member 118 may be a cylindrical or disc-shaped body coupled to each of actuators 110, 111. For example, actuator member 118 may include a cam or a wheel. In some examples, a central longitudinal axis 199 of medical device 100 may extend through actuator member 118.

FIG. 2B illustrates a magnified portion of handle 102 with a portion of handle 102 removed to expose interior components of handle 102. Actuation member 118 may be pivotably or rotatably coupled to the handle body 187 of handle 102 at a pivot portion 150, and actuation member 118 may be configured to pivot or rotate about pivot portion 150. In some examples, actuation member 118 may be part the handle body 187 of handle 102. Actuation member 118 may be cylindrical in shape and may have any suitable diameter; for example, actuation member 118 may have a diameter larger than half of the width of handle body 187 (as shown in FIG. 2B). Actuation member 118 may be positioned at a proximal portion of handle 102 to facilitate operating actuators 110, 111, for example, for operation by a user's index, middle finger, thumb, and/or any other fingers. Actuation member 118 may pivot or rotate about an axis transverse from central longitudinal axis 199, and in some examples, actuation member 118 may pivot or rotate about an axis normal to central longitudinal axis 199. For example, actuation member 118 may pivot or rotate about an axis extending through pivot portion 150 normally to longitudinal axis 199. As shown in FIG. 2B, a first half 141 of actuation member 118 may be thicker than a second half 142 of actuation member 118. Actuator 110 may include a cylindrical portion 161 (e.g., a stem) that may be pivotably or rotatably coupled to actuation member 118 at a pivot portion 162; and actuator 111 may include a cylindrical portion 161 (e.g., a stem) that may be pivotably or rotatably coupled to actuation member 118 at a pivot portion 163. In some examples, each of cylindrical portions 160, 161 may extend across second half 142 of actuation member 118. In alternatives, portions 160, 161 may have non-cylindrical shapes (e.g., rectangular prism, flat, or other shapes). In other examples, each of cylindrical portions 160, 161 may include a rolling wheel which rolls along a surface of the actuation member 118 (e.g. can slide or roll along a surface of actuation member 118 during actuation), and may not be pivotably coupled to actuation member 118.

In some examples, cylindrical portion 160 may be received within a recessed portion 164 of an expanded end portion 210 of actuator 110; and cylindrical portion 161 may be received within a recessed portion 165 of an expanded end portion 211 of actuator 111. Expanded end portion 210 of actuator 110 may be received within and configured to move within a recessed portion 170 of an exterior portion of handle body 187; and expanded end portion 211 of actuator 111 may be received within and configured to move within a recessed portion 171 of an exterior portion of handle body 187. Recessed portions 170, 171 may be configured to limit movement of expanded end portions 210, 211 in all directions except along a central longitudinal axis 298, 299, respectively, of the recessed portion 170, 171.

As shown in FIG. 2B, articulation wires 120, 121 may extend circumferentially around actuation member 118, and may be fixedly coupled to a peripheral portion of actuation member 118. FIG. 2B illustrates a neutral (or unactuated) position of actuators 110, 111 which corresponds to the distal articulating portion 106 being in a substantially straight (or unbent) position. In some examples, each of actuators 110, 111 may be biased towards the neutral position, for example by one or more biasing members such as a spring or other biasing member known in the art. In some examples, translation of actuator 110 along central longitudinal axis 298 may be limited by one or more stop features, such as a protruding portion of handle 102 or other translation limiting mechanism, to prevent over-bending of distal articulating portion 106. Also in some examples, translation of actuator 111 along central longitudinal axis 299 may be limited by one or more stop features, such as a protruding portion of handle 102 or other translation limiting mechanism, to prevent over-bending of distal articulating portion 106.

Rotation of actuation member 118 in a first direction may move articulation wire 120 proximally and articulation wire 121 distally, and articulation wire 120 may extend around a larger portion of the circumference of actuation member 118 relative to a neutral position (shown in FIG. 2B). Rotation of actuation member 118 in a second direction opposite from the first direction may move articulation wire 120 distally and articulation wire 121 proximally, and articulation wire 121 may extend around a larger portion of the circumference of actuation member 118 compared to a neutral position (shown in FIG. 2B). Each of actuators 110, 111 may be configured to rotate actuation member 118 when actuated.

For example, movement of actuator 110 in a first direction along central longitudinal axis 298 of actuator 110 may result in rotation of actuation member 118 in a second direction (e.g. rotation in a counter-clockwise direction about pivot portion 150 if facing actuation member 118 as shown in FIG. 2B). When actuator 110 is moved in this first direction (e.g. towards an opposite side 288 of handle 102 from recessed portions 170, 171 and/or towards central longitudinal axis 199), actuator 111 may be translated in an opposite direction along central longitudinal axis 299 (e.g. away from opposite side 288 of handle 102 along axis 299). Movement of actuator 110 may be achieved by a user pressing actuator 110 with one or more fingers or thumbs. Movement of actuator 111 in a third direction along central longitudinal axis 299 of actuator 111 may result in rotation of actuation member 118 in a fourth direction (e.g. rotation in a clockwise direction about pivot portion 150 if facing actuation member 118 as shown in FIG. 2B). When actuator 111 is moved in this third direction (e.g. towards an opposite side 288 of handle 102 from recessed portions 170, 171 and/or toward central longitudinal axis 199), actuator 110 may be translated in an opposite direction along central longitudinal axis 298 (e.g. away from opposite side 288 of handle 102 along axis 298). The first direction and the third direction may be substantially parallel to one another. The second direction and the fourth direction may be opposite rotational directions from one another.

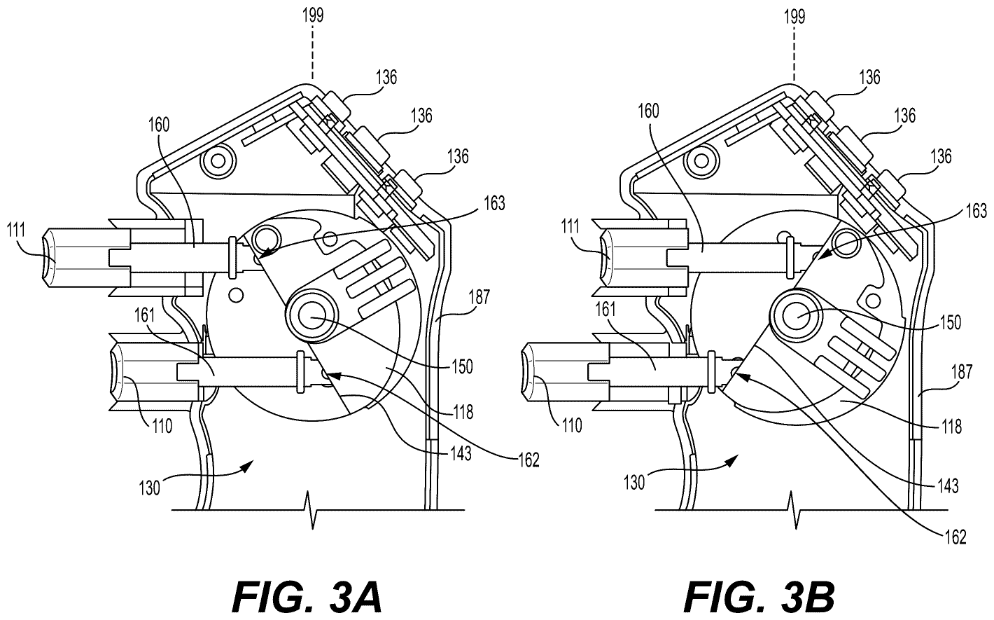

FIGS. 3A and 3B illustrate side views of a proximal portion of handle 102 with a portion of handle 102 and articulation wires 120, 121 removed. FIG. 3A illustrates a first position of actuators 110, 111 with actuator 110 in an actuated position, and FIG. 3B illustrates a second position of actuators 110, 111 with actuator 111 in an actuated position. As shown in FIGS. 3A and 3B, pivot portions or points 162, 163 are longitudinally offset (or staggered) from each other when either of actuators 110, 111 is in an actuated position (relative to a longitudinal axis of medical device 100), and pivot points 162, 163 are longitudinally aligned when actuators 110, 111 are in a neutral position (shown in FIG. 2B). FIG. 3A illustrates how actuation member 118 is rotated counter-clockwise (in the perspective of FIGS. 3A and 3B) relative to the neutral position when actuator 110 is actuated; and FIG. 3B illustrates how actuation member 118 is rotated clockwise (in the perspective of FIGS. 3A and 3B) relative to the neutral position when actuator 111 is actuated. You may want to mention somewhere that, when 110 moves inward, 111 moves outward, and vice versa. When 110 is actuated along its axis, 111 also technically moves along its axis, just in an opposite (outward) direction. Same for when 111 is actuated.

In some examples, the position of actuators 110, 111 shown in FIG. 3A may correspond to distal articulating portion 106 in a bent or curved upward position; and the position of actuators 110, 111 shown in FIG. 3B may correspond to distal articulating portion 106 in a bent or curved downward position. In other examples, the position of actuators 110, 111 shown in FIG. 3A may correspond to distal articulating portion 106 in a bent or curved laterally in a first direction or to the right; and the position of actuators 110, 111 shown in FIG. 3B may correspond to distal articulating portion 106 in a bent or curved laterally in a second direction opposite from the first direction or to the left.

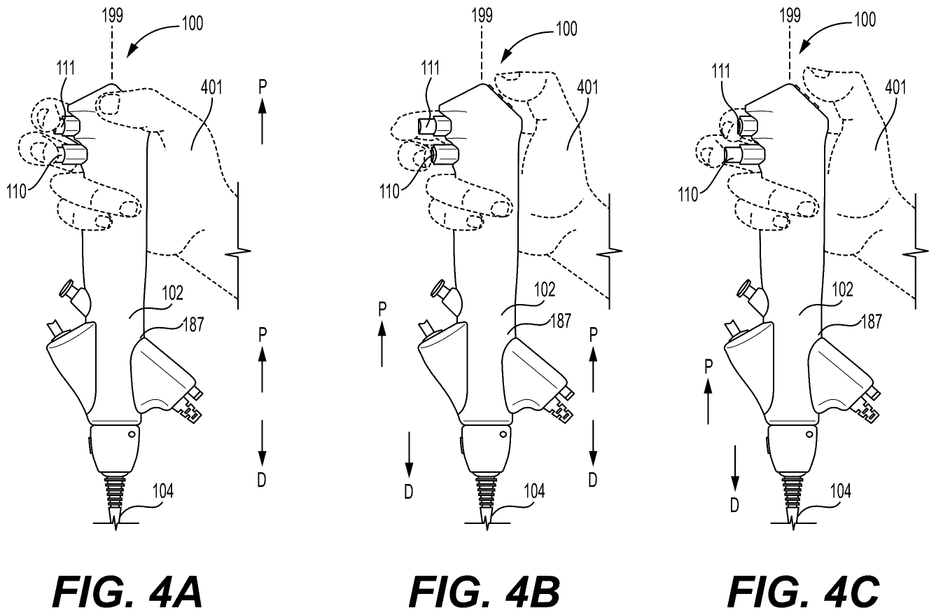

FIGS. 4A, 4B, and 4C illustrate medical device 100 with a user's hand 401 in various states of operation. Specifically, FIG. 4A illustrates medical device 100 in a neutral position with each of actuators 110, 111 not actuated (unactuated), and thus corresponds to a substantially straight position of distal articulating portion 106. FIG. 4B illustrates medical device 100 in a first actuated position with a middle finger of hand 401 actuating actuator 110 (e.g. pressing actuator 110), which corresponds to distal articulating portion 106 being bent in a first direction. FIG. 4C illustrates medical device 100 in a second actuated position with an index finger of hand 401 actuating actuator 111 (e.g. pressing actuator 111), which corresponds to distal articulating portion 106 being bent in a second direction opposite from the first direction. In other examples, actuators 110, 111 may not bend distal articulating portion 106 in opposite directions but may bend distal articulating portion 106 in two different directions. For example, actuator 110 may bend distal articulating portion 106 upward and actuator 111 may bend distal articulating portion 106 to the left. Actuators 110, 111 may bend distal articulating portion 106 in any combination of two directions.

Although FIGS. 4A, 4B, and 4C illustrate hand 401 operating actuators 110, 111 with an index finger and a middle finger, actuators 110, 111 may be operated by any combination of one or more fingers or thumbs. By positioning actuators 110, 111 at a proximal end of handle 102, the user may more easily access other components of medical device 100 during operation with the user's second hand (e.g. the hand not holding handle 102), such as working channel port 116 or umbilicus 108. Also, by positioning actuators 110, 111 proximate or adjacent to one or more buttons 136, a user may more easily access buttons 136 during actuation of actuators 110, 111, such as by accessing one or more buttons 136 with the user's thumb.

During an exemplary medical procedure, a user may move shaft 104 into a body lumen of a patient while actuators 110, 111 are in a neutral position and distal articulating portion 106 is substantially straight. The user may navigate a patient's one or more body lumens by bending distal articulating portion 106 via actuation of actuators 110, 111. The user may then treat the patient at a target site within the patient's body, for example by capturing images at the target site, applying suction and/or fluid at the target site, ablating tissue using one or more accessory devices, and/or collecting tissue samples with a grasper, basket, or other accessory device. The user may then remove medical device 100 from the body of the patient.

Medical device 100 including actuators 110, 111 may facility reducing user fatigue during operation of medical device 100, since a user may be required to move their fingers and/or thumb in a more ergonomic position compared to other actuators known in the art, such as a large wheel actuator. The design of actuators 110, 111 and their relative position on handle 102 may allow a user to more easily operate accessory devices while articulating shaft 104 of medical device 100, may reduce user error due to the increased ergonomics, and may simplify procedures by facilitating actuation of movement of distal articulating portion 106.

It will be apparent to those skilled in the art that various modifications and variations may be made in the disclosed devices and methods without departing from the scope of the disclosure. Other aspects of the disclosure will be apparent to those skilled in the art from consideration of the specification and practice of the features disclosed herein. It is intended that the specification and embodiments be considered as exemplary only.

We claim:

1. A medical device comprising:
a shaft including a distal articulating portion; and
a handle coupled to the shaft and comprising:
a first actuator moveably coupled to a handle body of the handle, wherein the first actuator is configured to initiate bending of the distal articulating portion in a first direction when the first actuator is translated along a first axis transverse from a central longitudinal axis of the medical device.

2. The medical device of claim 1, wherein the handle further comprises a second actuator moveably coupled to the handle body, wherein the second actuator is configured to initiate bending of the distal articulating portion in a second direction when the second actuator is translated along a second axis transverse from a central longitudinal axis of the medical device, wherein the second direction is different from the first direction.

3. The medical device of claim 2, wherein the first actuator and the second actuator are positioned at a proximal portion of the handle.

4. The medical device of claim 1, wherein the handle further comprises:
an actuation member pivotably coupled to a body of the handle, wherein the first actuator is pivotably coupled to the actuation member.

5. The medical device of claim 4, wherein the actuation member is cylindrical.

6. The medical device of claim 5, further comprising a first articulation wire coupled to the actuation member, wherein the first articulation wire extends longitudinally to a first portion of the distal articulating portion.

7. The medical device of claim 6, further comprising a second articulation wire coupled to the actuation member, wherein the second articulation wire extends longitudinally to a second portion of the distal articulating portion.

8. The medical device of claim 3, wherein the handle further comprises a third actuator positioned proximal of the first actuator and the second actuator.

9. The medical device of claim 1, wherein the first actuator includes a first portion and an expanded distal portion, wherein the first portion is positioned within the handle body and at least a portion of the expanded distal portion is external of the handle body.

10. The medical device of claim 9, wherein the first portion (i) is pivotably coupled to an actuation member or (ii) includes a rolling wheel which is configured to roll along a surface of the actuation member.

11. The medical device of claim 10, wherein translation of the first actuator along the first axis is configured to rotate the actuation member, and wherein the first portion is a cylindrical portion.

12. The medical device of claim 2, wherein the first actuator is received within a first recessed portion of a body of the handle, and the second actuator is received within a second recessed portion of the body of the handle.

13. The medical device of claim 2, wherein the handle further comprises an actuation member coupled to a body of the handle at a first pivot point; wherein the first actuator is pivotably coupled to the actuation member within the handle body at a second pivot point, and wherein the second actuator is pivotably coupled to the actuation member at a third pivot point on an opposite side of the first pivot point from the second pivot point.

14. The medical device of claim 13, wherein the second pivot point and the third pivot point are longitudinally aligned with a longitudinal axis of the medical device when the medical device is in a neutral position.

15. The medical device of claim 2, wherein the first actuator and the second actuator are each biased towards a neutral position.

16. A medical device comprising:
a shaft including a distal articulating portion; and
a first actuator, wherein the first actuator is configured to initiate bending of the distal articulating portion in a first direction when the first actuator is translated along a first axis transverse from a central longitudinal axis of the medical device.

17. The medical device of claim 16, further comprising an actuation member coupled to and/or abutting the first actuator, wherein the actuation member is cylindrical and coupled to an articulation wire;
wherein the articulation wire extends longitudinally through the shaft to the distal articulating portion.

18. The medical device of claim 17, further comprising a second actuator coupled to and/or abutting the actuation member, wherein the second actuator is configured to initiate bending of the distal articulating portion in a second direction when the second actuator is translated along a second axis transverse from a central longitudinal axis of the medical device, wherein the second direction is different from the first direction.

19. The medical device of claim 18, wherein the articulation wire is a first articulation wire, and the medical device further comprising a second articulation wire coupled to the actuation member and extending longitudinally through the shaft to the distal articulating portion.

20. A medical device comprising:
a shaft including a distal articulating portion;
an articulation wire extending longitudinally through the shaft and coupled to a portion of the distal articulating portion; and
a handle coupled to the shaft and comprising:
a handle body,
a first actuator, wherein the first actuator is configured to initiate bending of the distal articulating portion in a first direction when the first actuator is translated along a first axis, and
an articulation member pivotably coupled to the first actuator and pivotably coupled to the handle body, wherein actuation of the first actuator is configured to rotate the articulation member and move the articulation wire.
